(19) United States
(12) Patent Application Publication
(10) Pub. No.: US 2025/0043232 A1
(43) Pub. Date: Feb. 6, 2025

(54) MODEL-BASED ANALYTICAL TOOL FOR BIOREACTORS

(71) Applicant: MERCK PATENT GMBH, DARMSTADT (DE)

(72) Inventors: Laurent JOURDAINNE, Molsheim (FR); Adele SCHINI, Molsheim (FR); Karine Elise VOLTZ, Molsheim (FR); Bartolomeo DE CANDITIIS, Limonest (FR)

(73) Assignee: MERCK PATENT GMBH, DARMSTADT (DE)

(21) Appl. No.: 18/715,545

(22) PCT Filed: Dec. 1, 2022
(86) PCT No.: PCT/EP2022/084079

§ 371 Date: May 31, 2024

(30) Foreign Application Priority Data
Dec. 2, 2021 (EP) 21306688.9

(51) Int. Cl. C12M 1/36; C12M 1/34

(52) U.S. Cl. CPC C12M 41/48; C12M 1/3407; C12M 1/36; C12M 41/36

(57) ABSTRACT

Method and system to analyze biomasses in a bioreactor (3) via a computer (2) with a system software (5), the bioreactor (3) having at least one sensor (6) to measure the biomasses and which has a data connection to the computer (2) managed by a data interface provided by the system software (5), wherein the system software (5) provides a data conversion model (8) to analyze real time raw data about permittivity measured by and transmitted from the at least one sensor (6) to the computer (2) to calculate specific cell parameters of cells in the biomasses.

The hereby described invention discloses a method to operate an in situ analytical tool in bioreactors using a computer supported physics-based model.

### TECHNICAL FIELD

The invention deals with the technological area of a continuous biopharmaceutical process.

### BACKGROUND AND DESCRIPTION OF THE PRIOR ART

The pharmaceutical industry's quality approach is focused on improving and increasing productivity in the manufacture of biochemical compounds. This requires the use of complex bioprocesses with real-time monitoring integrated within the production line. In-line analysis can enable the process automation, thus optimizing it by a significant saving of time and materials. Currently, there is a wide range of sensors and off-line technologies on the market capable of monitoring essential variables in a cell culture, like biomass, radius, nutrient quantity, metabolic indicators etc, as well as critical parameters of a bioprocess, but few of them are converted into in-situ sensors.

The conversion of analytical tools towards in situ sensors is therefore a current exploratory trend, aiming at improving the quality of their measurements. Moreover, thanks to these optimized sensors, called Process Analytical Tools (PAT), the conditions of continuous or discontinuous cell cultures could be adjusted in real time thanks to physical measurements converted through models to quantitative and qualitative information. This adaptation to in-line sensors has a lot of benefits: no cleaning steps, less system downtime, no cleanroom requirement, and reduced costs.

Another tendency is the conversion from multi-use (MU) to single-use (SU) sensors which provide similar advantages, especially the missing necessity of cleaning steps. Unfortunately SU sensors have a major difficulty about their calibration which cannot be performed prior to the system installation.

These process sensors and analytical tools require therefore specific and complex calibration models based on a large amount of data to handle those difficulties.

Summarized there are four major problem statements regarding the mentioned known state of the art:
1. Usually at the first use in a specific application and process, PAT only deliver raw data and don't directly give parameters information and measurements like viable cell density, glucose concentration etc. For instance, the dielectric spectroscopy gives quantitative media permittivity data but no viable cell density data. Thus, a complex conversion or calibration model must be developed.
2. Data driven-based calibration models for PAT are the preferred models as no other approach seems to be currently implemented and used in this field of application. Data driven-based calibration models for PAT require several cell culture runs and a large amount of data to give parameter measurements with acceptable accuracies and measurement tolerances.
3. Amongst the difficulties to convert multi-use sensors or PAT in single-use versions, a major difficulty with SU variations is their specific calibration, quite different from the MU sensor calibration. Whereas process MU sensors can be offline calibrated right before the run, process SU sensors require pre-calibration data given by the supplier. Data driven-based calibration models for analytical tools are impossible to transfer from a MU to another MU or a SU PAT because a part of the model is probe dependent, like specific sensitivity for instance, internal factory coefficient.
4. The scale up from e.g. a 3 L bioreactor to a significant larger bioreactor, e.g. 2 kL, is a challenge for in-situ analytics as their model are data driven based. These data can be sensitive to the size of the bioreactor and the condition of culture that can be quite different with the volume, like mixing, sparging etc.

### SUMMARY OF THE INVENTION

The task of this patent application is therefore to find a method to use an analytical tool in bioreactors which can overcome the known limitations of the prior art.

This task has been solved by a method to analyze biomasses in a bioreactor via a computer with a system software, the bioreactor having at least one sensor to measure the biomasses and which has a data connection to the computer managed by a data interface provided by the system software, wherein the system software provides a data conversion model to analyze real time raw data about permittivity measured by and transmitted from the at least one sensor to the computer to calculate specific cell parameters of cells in the biomasses. The purpose of the invention is the transformation of the sensor, in this case a capacitance probe, integrating the dielectric spectroscopy into a true biomass probe providing qualitative and quantitative information on cell parameters, like radius and viable cell density. Important is further that the probe works in real time to provide the raw data, with a reduced effort of calibration, and for either multi-use or single-use probe variations. This approach solves the four described problems one by one:

### Problems 1 & 2

The physics-based model is usable from the very first use of the probe and does not require any machine learning and/or model building as parameters and coefficients of the model, because this data are either coming from the probe measurements, are extrapolated from offline measurements or leveraged from the literature. The physics-based model also does not require a large amount of data nor prior calibration-based on older cell culture runs as it is based on equations describing cells as “dielectric” objects. It is able to use real-time physical values taken from the probe.

### Problem 3

The physics-based model is sensor independent and factory calibration-free. The model can therefore self-calibrate with the used sensor. Parameters to be extracted from the equations are coming from cells considered as dielectric objects, and thus the model can be transferred from one multi-use probe to another MU one, or a single-use probe.

### Problem 4

The physics-based model is cell line independent while the cells have the shape modelized in the model. Indeed, as cells are considered as dielectric object, thus their biochemical specificities are not a root cause of interference in the model.

The cell membrane capacitance Cₘ and the internal conductivity σᵢ are calculated from an offline analysis and allow the regular adjustment of the model while giving qualitative information of the cell.

Preferred further developments of the process include, for example, but are not limited to that:
additionally to using the mere physics-based data model to analyze the real time raw data a data driven machine learning approach is used for the data conversion model resulting in a hybrid data conversion model with improved accuracy.
the at least one sensor measures amplitudes of the permittivity at various excitation frequencies as real time raw data.
the computer calculates as cell parameters the cell dimension in form of its radius or diameter and a viable cell density (VCD) in consideration of predefined parameter values of cell membrane capacitance and internal conductivity.
the data is discontinuously adjusted based on sampling and offline analysis of the cell membrane capacitance and internal conductivity.
an averaged value of the cell membrane capacitance and internal conductivity is calculated via offline analyses after the end of every measurement turn and used for following measurement turns instead of the previously defined parameter values.

Another solution to this task is an automated system for analyzing biomasses comprising a bioreactor with at least one sensor to measure the biomasses, a computer being connected to the at least one sensors and a system software performed on the computer with a data interface managing the connection to the at least one sensor and providing a data conversion model, being arranged to perform the previously described method.

Preferred further developments of the automated system include, for example, but are not limited to that:
the at least one sensor is a capacitance probe integrating dielectric spectroscopy.
the software comprises a specific software module implemented between the smart dielectric spectroscopy probe and the data interface which enables the real time raw data processing with the embedded model.
the at least one sensor is a disposable single-use sensor.
the computer is a single control unit which performs the system software and the data conversion model.
the computer comprises a first computer being connected to the at least one sensors which controls the bioreactor and performs the system software with a data interface managing the connection to the at least one sensor and a second computer at a remote location which provides the data conversion model and uses a connection to the first computer via its data interface.
the data conversion model is independent of the at least one sensor being a single-use or multi-use probe and can be used for separate sensors, meaning that the model is used for more than one sensor, be it multi- or single-use.

### DETAILED DESCRIPTION OF THE INVENTION

The method and the automated system 1 including the software 5 according to the invention and functionally advantageous developments of those are described in more detail below with reference to the associated drawings using at least one preferred exemplary embodiment. In the drawings, elements that correspond to one another are provided with the same reference numerals.

The drawings show:

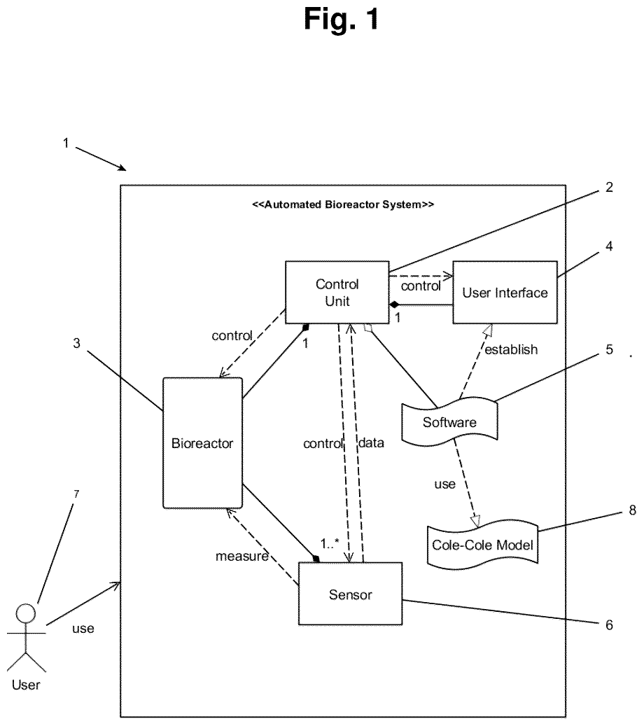

FIG. 1: a schematic overview about the used automated bioreactor system

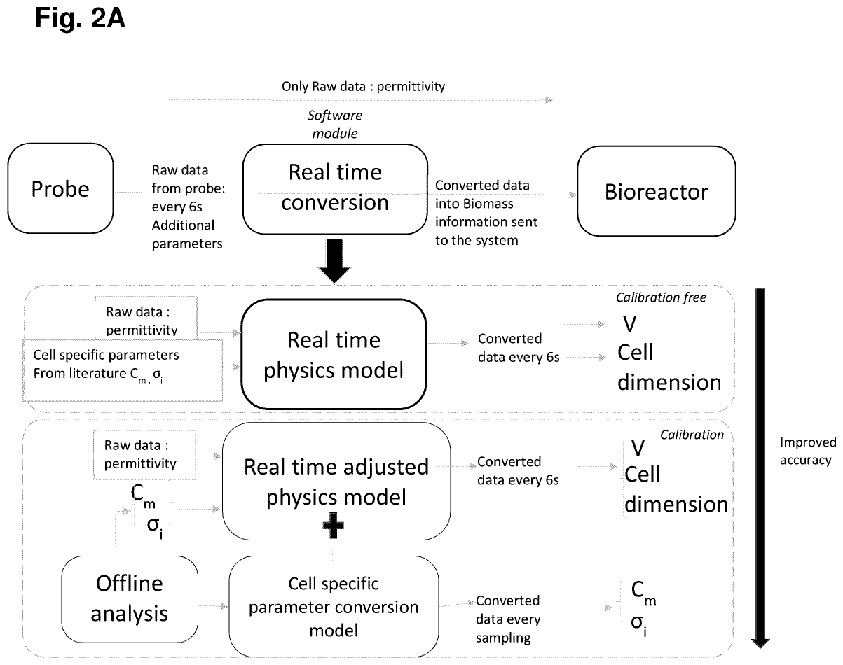

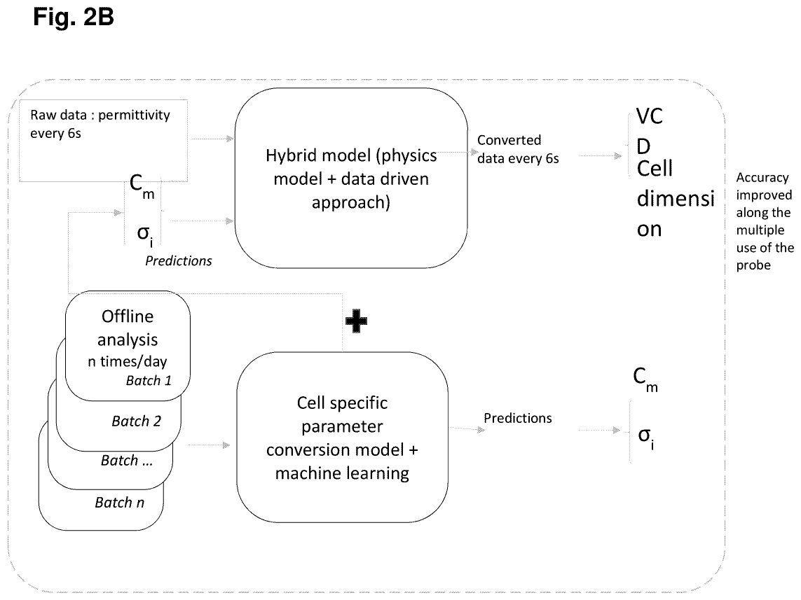

FIG. 2: a comprehended schematic overview about the different preferred embodiments of the used model

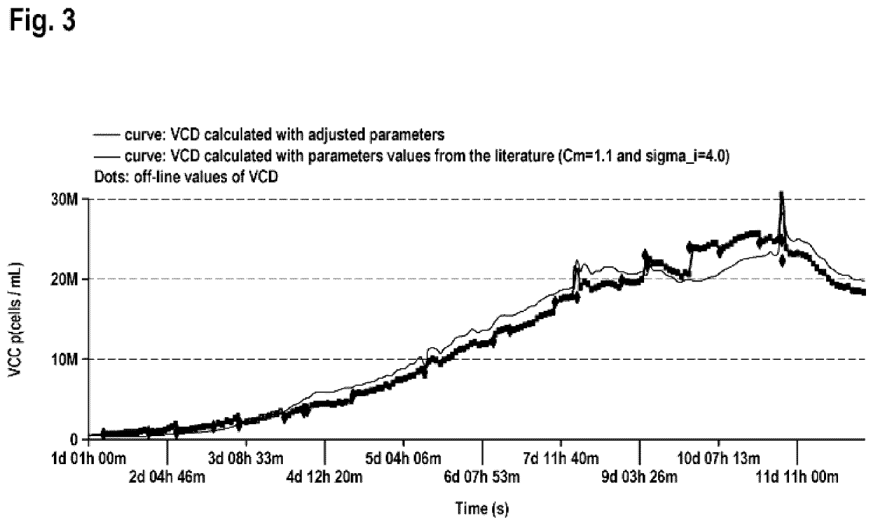

FIG. 3: result curves for the viable cell density (VCD)

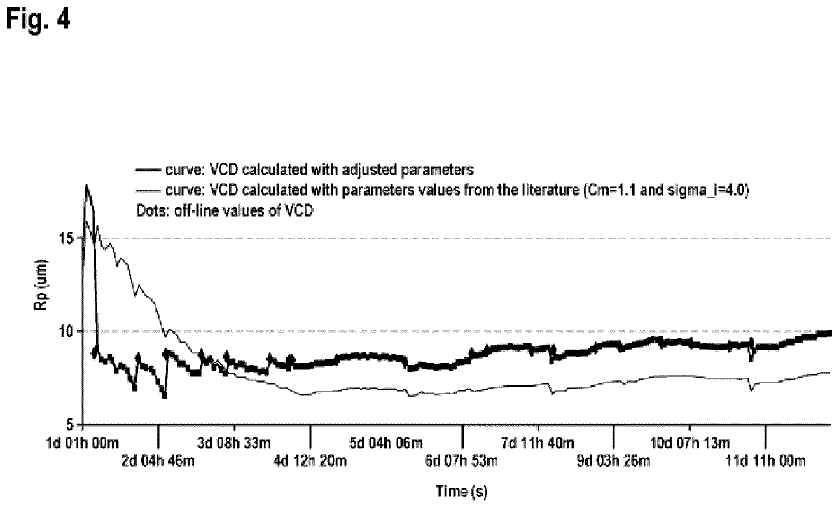

FIG. 4: result curves for the radius (R)

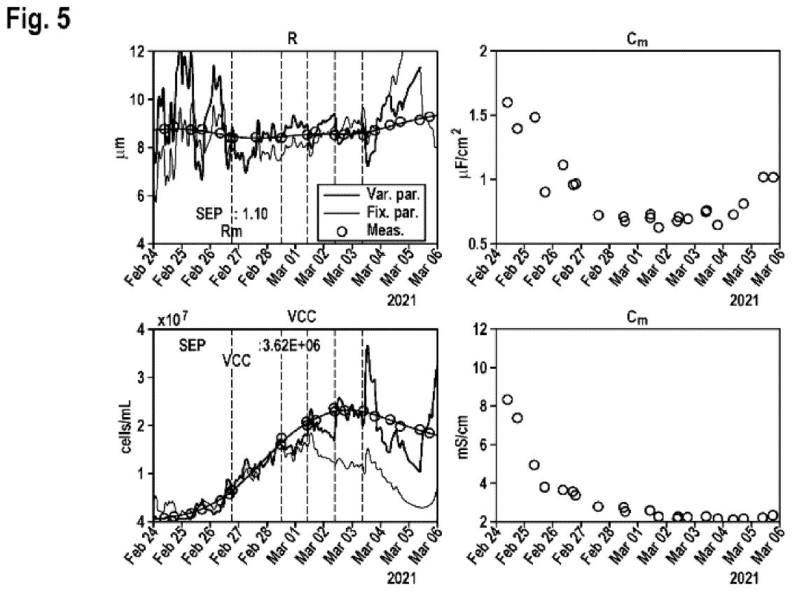

FIG. 5: averaged value for cell membrane capacitance and internal conductivity

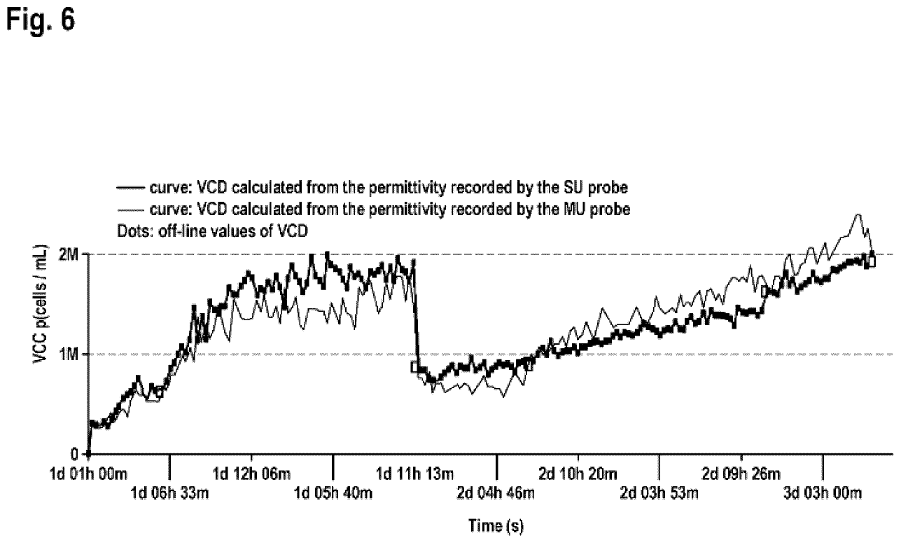

FIG. 6: respective result curves for the viable cell density (VCD) compared for single-use and multi-use probes

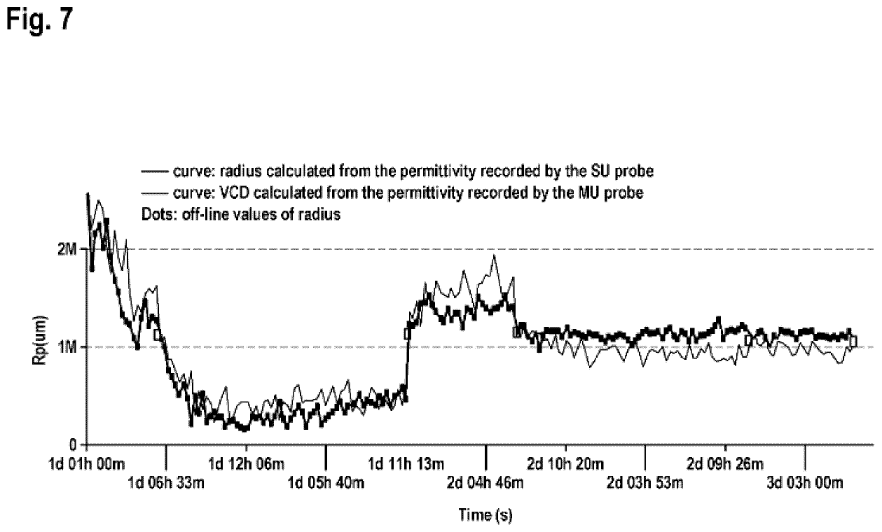

FIG. 7: respective result curves for the and radius (R) indications compared for single-use and multi-use probes

FIG. 1 shows an example of an automated bioreactor system 1 which is used for the invention. It comprises of the bioreactor 3 itself which contains a biomass with cell cultures, its control unit 2, a biomass sensor 6 connected to the bioreactor 3 and a system software 5 run by the control unit 2 which uses a specific data model 8 to calculate specific cell parameters of the cells in the biomass, by analyzing real time raw data about permittivity measured by and transmitted from the at least one sensor 6 to the control unit 2. The control unit 2 is preferably a standard computer suitable to control the bioreactor 3. Another option is a microcontroller or a processor integrated in an embedded device with the bioreactor 3. It could also be a standard or industrial personal computer or server or any other suitable device, especially if the local control unit 2 provides the data model 8 itself, because then a higher processing power as usually provided by a microcontroller is required. In another preferred embodiment the data model 8 is provided by a suitable separate computer at a remote location via a data network using a cloud-based service.

The data model 8 is preferably a phenomenological Cole-Cole model 8 which convert real time raw data of permittivity into viable cell density (VCD) and average cell culture radius (R) indications. Based itself on the Debye equation (Debye, 1929), the Cole-Cole equation reproduce the shape of the β-dispersion by expressing the permittivity (ε) as a function of frequency (f) and can be written as follows:

$$ε ( f ) = \frac{Δ ε [ 1 + {( f / {f}_{c} )}^{1 - α} sin ( α π / 2 ) ]}{1 + {( f / {f}_{c} )}^{2 ( 1 - α )} + 2 {( f / {f}_{c} )}^{1 - α} sin ( α π / 2 )} + {ε}_{0} {ε}_{∞}$$

where Δε is the amplitude of the distribution, f_{c} is the characteristic frequency (that is the frequency at which & equals half the value of Δε), α is the slope of the distribution, ε₀ is the permittivity of free space, and ε_{∞} is the permittivity at high frequency (usually above 1 MHZ) [Opel et al., 2010].

The dielectric parameters Δε, fc, and α are calculated by the INCYTE internal software (ArcAir, Hamilton) from raw permittivity data each time a scan is executed.

The Cole-Cole parameters can be linked to quantitative information of the cells, like the average culture cell radius R by using the following equations:

$$R = \frac{1}{2 π {f}_{c} {C}_{m} ( \frac{1}{{σ}_{i}} + \frac{1}{2 {σ}_{a}} )}$$

where Cₘ (measured in F/m²) and σᵢ (measured in S/m) are respectively the average membrane capacitance and the internal conductivity of cells in the culture. The quantity σₐ (measured in S/m) represent the static medium conductivity and can be determined from the equation:

$${σ}_{a} = \frac{σ}{{( 1 - {p}_{p} )}^{1.5}}$$

where σ (measured in S/m) is the static suspension conductivity, and pₚ is the predicted biomass volume fraction expressed in the following way:

$${p}_{p} = \frac{4 Δ ε}{9 r {C}_{m}}$$

Finally the viable cell density VCD is calculated starting from the assumption that the cells in the culture are spherical, thus the single cell volume V can be written as:

$$V = \frac{4}{3} π {R}^{3}$$

and therefore:

$$VCD = \frac{{p}_{p}}{V} = \frac{Δ ε}{3 π {R}^{4} {C}_{m}}$$

The software 5 which provides and applies the Cole-Cole model 8 also comprises a raw data conversion module. In its graphical user interface (GUI) 4, the user 7 can choose the type of modeling he wants to use for the calculations. Preferably the MATLAB software (The MathWorks Inc) is used as software 5, but any other suitable software can also be used. In this example MATLAB version 9.9.0.1570001 from 2020 was used.

Using that model 8 in an algorithm, r and VCD values were calculated every minutes. Two daily samples were taken to obtain average offline values of cell radius and VCD. They were interpolated with a smoothing spline. The values calculated by the model 8 were compared to the spline and the Standard Error Prediction (SEP) was calculated, as follow:

$$SEP = \sqrt{\sum \frac{{( \hat{y} - y )}^{2}}{{n}^{p}}}$$

The computer software 5 is preferably integrated on an platform to monitor radius and VCD during cultivation. Using this GUI 4, the user is requested to enter theoretical values for Cₘ and σᵢ as well as files containing raw permittivity values. It is also possible, depending on the chosen model 8, to add a file containing the values determined offline with the Nova analyzer. The raw permittivity data could also be provided in an alternative option by the biomass sensor 6 in real-time.

The calculated radius and VCD values will be compared to offline measurements made with an automated cell culture analyzer. By doing so the validity of the Cole-Cole model 8 applied to cells in culture is tested.

The specific software module is preferably implemented in the system software in between the smart dielectric spectroscopy probe and the software interface and enables the real time raw data processing with the embedded model 8.

The following method steps show a preferred example to use the model 8 with the best accuracy:
1) use of the described pure physics-based Cole-Cole model 8 with the probe 6 delivering real time permittivity measurements at various excitation frequencies. Real time means one measurement every six seconds at the fastest. The probe 6 is directly used as a biomass sensor 6 from the very first field use of it with cell specific parameters, preferably the cell membrane capacitance and internal conductivity, taken from the literature. That can be used up to the two or three first days of the cell culture in the bioreactor 3. FIGS. 3 and 4 show result curves for the viable cell density (VCD) and radius (R) indications.
2) Discontinuous adjustment of the conversion model 8 based on sampling and offline analysis of cell membrane capacitance and internal conductivity. The model 8 opens the calculation at each sampling of these cell specific parameters based on the following equations:

$$Cm = \frac{3 Δ ε}{π {r}^{4} {N}_{v}}$$

$${σ}_{i} = \frac{1}{\frac{1}{{f}_{c} {rC}_{m}} - \frac{1}{2 {σ}_{e}}}$$

FIG. 5 shows an averaged value of each of these two cell specific parameters which can be calculated after the end of the run and used later instead of literature parameter values.
3) The model is transferable to a disposable, single-use sensor without any specific sensor adjustment as experimental data show. FIGS. 6 and 7 show the respective result curves for the viable cell density (VCD) and radius (R) indications.

As conclusion, it can be comprehended that the adjusted model 8 can be used either on MU or SU probes 6 without any additional calibration step on the SU sensor as usually required on typical process control sensors, like pH, dissolved oxygen, while not losing the calibration-free feature of the invention. The scalability to characterize and monitor cell cultures from small to large bioreactor is obvious as the model 8 is cell line independent and uses cells as dielectric objects. Improving the accuracy of the model 8 is done with a data driven approach combined with the physics-based model 8 giving a hybrid model. FIG. 2 gives a comprehended schematic overview about the invention including the different preferred embodiments of the used model 8.

### LIST OF REFERENCES

1 Automated bioreactor system
2 Control unit/computer
3 Bioreactor
4 User interface
5 Software
6 Sensor/Probe
7 User
8 Data Conversion Model (Cole-Cole)

1. A method to analyze biomasses in a bioreactor (3) via a computer (2) with a system software (5), the bioreactor (3) having at least one sensor (6) to measure the biomasses and which has a data connection to the computer (2) managed by a data interface provided by the system software (5), wherein
the system software (5) provides a data conversion model (8) to analyze real time raw data about permittivity measured by and transmitted from the at least one sensor (6) to the computer (2) to calculate specific cell parameters of cells in the biomasses.

2. The method according to claim 1, wherein a physics-based data model based on Cole-Cole equations is used as a data conversion model (8).

3. The method according to claim 2, wherein additionally to using the mere physics-based data model to analyze the real time raw data a data driven machine learning approach is used for the data conversion model (8) resulting in a hybrid data conversion model with improved accuracy.

4. The method according to claim 1, wherein the at least one sensor (6) measures amplitudes of the permittivity at various excitation frequencies as real time raw data.

5. The method according to claim 1, wherein the computer (2) calculates as cell parameters a cell dimension in form of cell radius or diameter and a viable cell density (VCD) in consideration of predefined parameter values of cell membrane capacitance and internal conductivity.

6. The method according to claim 5, wherein the data is discontinuously adjusted based on sampling and offline analysis of the cell membrane capacitance and internal conductivity.

7. The method according to claim 6, wherein an averaged value of the cell membrane capacitance and internal conductivity is calculated via offline analyses after the end of every measurement turn and used for following measurement turns instead of the previously defined parameter values.

8. An automated system for analyzing biomasses comprising a bioreactor (3) with at least one sensor (6) to measure the biomasses, a computer (2) being connected to the at least one sensors (6) and a system software (5) performed on the computer (2) with a data interface managing the connection to the at least one sensor (6) and providing a data conversion model (8), being arranged to perform one of the previous claims.

9. The automated system according to claim 8, wherein the at least one sensor (6) is a capacitance probe integrating dielectric spectroscopy.

10. The automated system according to claim 9, wherein the system software (5) comprises a specific software module implemented between the dielectric spectroscopy probe and the data interface which enables the real time raw data processing with the data conversion model (8).

11. The automated system according to claim 8, wherein the at least one sensor (6) is a disposable single-use sensor.

12. The automated system according to claim 8, wherein the computer (2) is a single control unit which performs the system software (5) and the data conversion model (8).

13. The automated system according to claim 8, wherein the computer (2) comprises a first computer being connected to the at least one sensors (6) which controls the bioreactor (3) and performs the system software (5) with a data interface managing the connection to the at least one sensor (6) and a second computer at a remote location which provides the data conversion model (8) and uses a connection to the first computer via a data network to the first computers data interface.

14. The automated system according to claim 8, wherein the data conversion model (8) is independent of the at least one sensor (6) being a single-use or multi-use probe and can be used for separate sensors.
